(19) United States
(12) Patent Application Publication
(10) Pub. No.: US 2026/0256363 A1
(43) Pub. Date: Sep. 3, 2026

(54) SYSTEMS AND METHODS FOR NON-INVASIVE, NON-CONTACT PHYSIOLOGICAL MONITORING, CARDIOVASCULAR PARAMETER ESTIMATION, AND WELLNESS INDICATION GENERATION FROM FACIAL VIDEO

(71) Applicant: BRGX Technologies LLC, Chattanooga,, TN (US)

(72) Inventors: Julian Gerald Dcruz, Kollam (IN); Ted Huang, Taipei (TW); Pai-chang Yeh, Zhubei (TW)

(73) Assignee: BRGX Technologies LLC, Chattanooga,, TN (US)

(21) Appl. No.: 19/656,801

(22) Filed: Apr. 23, 2026

Related U.S. Application Data
(63) Continuation-in-part of application No. 17/729,523, filed on Apr. 26, 2022, now Pat. No. 12,646,625.

(51) Int. Cl. A61B 5/0205; A61B 5/00; G06T 7/00; G06T 7/11

(52) U.S. Cl. CPC A61B 5/0205; A61B 5/0002; A61B 5/742; G06T 7/0012; G06T 7/11; G06T 2207/10101; G06T 2207/20081; G06T 2207/30201

(57) ABSTRACT

Systems, methods, apparatuses, and non-transitory computer-readable media are disclosed for non-contact physiological monitoring from facial video. A monocular RGB camera captures facial video of a subject under ambient lighting. One or more processors detect facial skin regions of interest by segmenting facial landmarks and computing a temporal imaging photoplethysmography signal-quality metric to select stable pulsatile regions. Imaging photoplethysmography signals and temporal features are extracted from the selected regions, and a trained convolutional neural network infers OCT-variation feature maps from RGB-video time series without using physical OCT hardware during inference. The processors combine the iPPG features, temporal features, OCT-variation feature maps, and motion-derived imaging ballistocardiography (iBCG)—facial micro-motion features to generate volumetric tensors. A trained model predicts pulse rate and additional physiological parameters, estimates uncertainty, outputs interpretability cues identifying contributing regions, and may generate evidence-grounded physiological wellness indications using retrieved contextual knowledge.

### RELATED APPLICATIONS

This application is directed to subject matter that builds upon, and in certain embodiments claims priority or benefit from U.S. patent application Ser. No. 17/729,523, titled “SYSTEM, METHOD AND APPARATUS FOR NON-INVASIVE & NON-CONTACT MONITORING OF HEALTH CHARACTERISTICS USING ARTIFICIAL INTELLIGENCE (AI),” filed April 26, 2022;. The foregoing application is incorporated herein by reference to the extent permitted and to the extent not inconsistent with the present disclosure.

### TECHNICAL FIELD

The present disclosure relates generally to computer-implemented, camera-based physiological monitoring. More particularly, the present disclosure relates to systems, methods, apparatuses, and non-transitory computer-readable media for estimating a subject's pulse rate from monocular RGB facial video, extracting imaging photoplethysmography (iPPG) and imaging ballistocardiography (iBCG) features, generating OCT-variation feature maps without use of physical OCT hardware during inference, estimating additional cardiovascular and respiratory parameters, quantifying model uncertainty, and generating evidence-grounded physiological wellness indications and recommendations.

### BACKGROUND

The following discussion is provided to facilitate understanding of the disclosed subject matter. It is not an admission that any discussion herein constitutes prior art to the presently claimed inventions.

Conventional physiological monitoring often depends on contact sensors such as electrocardiography electrodes, finger photoplethysmography probes, oscillometric cuffs, chest straps, and adhesive or wearable patches. Although clinically useful, such sensors can be uncomfortable, require user compliance, and are poorly suited to opportunistic measurements during driving, remote care, fitness activities, consumer-device use, or short telehealth encounters.

Remote photoplethysmography and imaging photoplethysmography have shown that subtle color variations in skin regions of a facial video can be used to recover periodic pulsatile information. However, many conventional video-based approaches rely on spatial averaging, narrow handcrafted filters, or a single preferred facial region, and can degrade under pose changes, illumination changes, partial occlusion, or subject motion.

At the same time, cardiovascular state is not fully characterized by volumetric blood-volume information alone. Mechanical recoil information associated with cardiac ejection, ventricular filling, and pulse propagation can provide complementary information relevant to heart-rate variability, respiratory coupling, blood-pressure dynamics, and overall hemodynamic state. A need therefore exists for a camera-based approach that combines optical and mechanical surrogates extracted from the same facial video stream.

A further need exists for technical mechanisms that select stable pulsatile regions of interest rather than treating all facial pixels as equally informative, that derive interpretable feature representations rather than opaque scalar outputs alone, and that preserve compatibility with low-cost monocular RGB cameras operating in ordinary ambient illumination.

There is also a need for a practical downstream reasoning layer. Raw physiological outputs such as pulse rate, heart-rate variability, respiratory rate, blood oxygen saturation, and blood-pressure trends are useful, but users and clinicians often need higher-level, evidence-grounded indications regarding stress, fatigue, autonomic recovery, nervous behavior, and activity state. Pure large-language-model generation without grounded retrieval can hallucinate, and pure rule systems can be brittle. A need therefore exists for a hybrid wellness reasoning framework that converts measured facial and physiological features into normalized textual contexts and retrieves supporting evidence from a curated knowledge base before generating recommendations or explanatory indications.

### SUMMARY

In one aspect, the present disclosure provides a computer-implemented method for non-invasive estimation of a subject's pulse rate using a monocular RGB video camera. The method may comprise capturing a sequence of facial video frames of the subject using a monocular RGB camera operating in a range of about 30 to 60 frames per second under ambient lighting; detecting candidate skin regions of interest (ROIs) by segmenting facial landmarks and regions using geometric heuristics or machine learning, and computing a temporal iPPG signal quality metric to select stable pulsatile ROIs; extracting iPPG signals and temporal features from the selected ROIs, the temporal features including pulse waveform amplitude, harmonics, and temporal signal-to-noise ratio; inferring an OCT-variation feature map for each selected ROI by executing a trained convolutional neural network on the facial video frames, wherein the trained convolutional neural network has been trained to map RGB-video time series to features that emulate structural or coherence-depth patterns characteristic of OCT imaging, without using interferometric OCT hardware during inference; combining the iPPG signals, the temporal features, and the OCT-variation feature map to generate a fused feature vector; predicting a pulse rate value from the fused feature vector; and outputting the pulse rate value together with an interpretability cue identifying at least one ROI used for inference. In preferred embodiments, no physical OCT sensor is used in any step performed during inference.

In another aspect, the selected ROIs may further be processed to extract motion-derived features, including imaging ballistocardiography signals and facial micro-motion features. In certain embodiments, the fused feature vector is represented as or derived from a volumetric tensor in which one dimension corresponds to temporal progression across facial video frames, two dimensions correspond to spatial coordinates of at least one selected ROI, and one or more feature channels correspond to iPPG signals, temporal features, motion-derived features, and OCT-variation feature maps.

In another aspect, the trained model may comprise convolutional layers and transformer layers including self-attention and positional encoding configured to process the volumetric tensor to capture spatiotemporal dependencies across the facial video frames. In various embodiments, the model is trained using facial video sequences correlated with ground-truth physiological signals selected from electrocardiogram data, photoplethysmography data, pulse oximetry data, respiratory references, cuff-based blood-pressure references, arterial waveform references, and subdermal proxy feature signals.

In another aspect, the disclosed system optionally estimates an uncertainty metric associated with one or more predicted physiological parameters and outputs the uncertainty metric together with the predicted values. In additional embodiments, the trained model is further configured to predict at least one additional physiological parameter selected from heart-rate variability, respiration rate, blood oxygen saturation, and blood pressure.

In still another aspect, latent variables generated by the spatiotemporal model are converted into physiological wellness indications. In one implementation, latent features and measured outputs are transformed into textual contexts that describe the subject's physiological state, facial appearance state, and user context. Those textual contexts are then used to retrieve evidence from a curated knowledge base, such as a graph-augmented retrieval system, and a medical or wellness language model generates grounded indications and recommendations relating to stress/anxiety, fatigue, nervous behavior, activity monitoring, recovery support, routines, diet, and supplements.

In another aspect, the present disclosure provides a non-contact, non-invasive physiological monitoring apparatus comprising a monocular RGB video camera, one or more processors, and a memory storing instructions that, when executed, cause the processors to perform one or more of the foregoing functions. In a further aspect, the present disclosure provides a non-transitory computer-readable medium storing instructions that, when executed by one or more processors operatively coupled to a monocular RGB video camera, cause the processors to perform one or more of the foregoing operations.

### BRIEF DESCRIPTION OF THE DRAWINGS

FIG. 1 illustrates an overall architecture of a non-contact physiological monitoring system including a monocular RGB video camera, an acquisition and quality-control pipeline, a feature fusion subsystem, a physiological prediction subsystem and a wellness retrieval subsystem-the prediction unit 106, and an output subsystem, in accordance with one embodiment of the present disclosure.

FIG. 2 illustrates an exemplary data-flow diagram in which facial video frames are converted into selected stable pulsatile ROIs, iPPG features, iBCG and facial micro-motion features, OCT-variation feature maps, fused feature vectors, predicted physiological parameters, and wellness indications, in accordance with one embodiment of the present disclosure.

FIG. 3 illustrates an exemplary mathematical and algorithmic framework for temporal iPPG quality scoring, feature fusion, uncertainty estimation, and prediction of pulse rate and additional physiological parameters, in accordance with one embodiment of the present disclosure.

FIG. 4 illustrates an exemplary feature-construction module in which optical, mechanical, and contextual facial features are assembled into a fused vector and/or volumetric tensor for downstream inference, in accordance with one embodiment of the present disclosure.

FIG. 5 illustrates an exemplary monitoring and knowledge-service configuration in which predicted physiological parameters and textual contexts are transmitted to a knowledge retrieval service configured to query a curated graph-based and vector-based knowledge base, in accordance with one embodiment of the present disclosure.

FIG. 6 illustrates an exemplary driver-monitoring embodiment in which a driver-facing monocular RGB camera operates as part of a vehicle or fleet-monitoring system.

FIG. 7 illustrates an exemplary fitness or activity-monitoring embodiment in which the disclosed system operates in connection with exercise equipment, a smartphone, or a consumer wellness device.

FIG. 8 illustrates an exemplary home or consumer-device embodiment in which the disclosed system is integrated into a display device, a smart mirror, or a personal-monitoring station.

FIG. 9 illustrates an exemplary telehealth embodiment in which the disclosed system operates through a client device or care portal to provide real-time physiological and wellness indications during a remote encounter.

FIGS. 10A-10C illustrate an exemplary method for non-invasive physiological monitoring and wellness indication generation from facial video.

### DETAILED DESCRIPTION

The following description describes exemplary embodiments and implementations in sufficient detail to enable a person of ordinary skill in the art to make and use the disclosed subject matter. The embodiments are illustrative rather than limiting, and features described in connection with one embodiment may be used in combination with features described in connection with another embodiment unless the context indicates otherwise.

### A. Non-Limiting Definitions

Monocular RGB video camera: a single-lens red-green-blue camera configured to capture a time sequence of facial video frames of a subject;

Region of interest (ROI): a facial skin region selected for physiological analysis, such as a forehead, cheek, perinasal, malar, chin, jawline, or other facial skin region;

Temporal iPPG signal quality metric: a score or vector of scores computed over a candidate ROI to quantify whether that ROI exhibits stable pulsatile behavior consistent with an imaging photoplethysmography waveform;

Temporal features: features derived from one or more ROI signals over time, including pulse waveform amplitude, harmonic content, temporal signal-to-noise ratio, pulse regularity, upstroke timing, inter-beat interval consistency, or derivatives thereof;

OCT-variation feature map: a spatially arranged learned representation inferred from RGB facial video by a trained neural network and configured to emulate structural or coherence-depth patterns characteristic of OCT imaging without requiring interferometric OCT hardware during inference;

Motion-derived features: features derived from facial motion and sub-pixel displacement, including iBCG signals, optical-flow trajectories, displacement, velocity, acceleration, periodic motion energy, phase lags, and mechanically informative latent features;

Volumetric tensor: a multi-dimensional feature representation having a temporal axis, spatial axes, and one or more feature channels for optical, mechanical, and contextual information;

Interpretability cue: a graphical, textual, or other indicator identifying one or more ROIs, temporal windows, or feature components used for inference, such as an ROI outline, heat map, confidence overlay, ranked ROI list, or attention map;

Subdermal proxy feature signals: signals or targets used during training to represent subsurface or physiological structure, such as OCT-derived targets, ultrasound-derived targets, perfusion proxy maps, vessel-depth proxy maps, pulse-wave propagation proxies, or model-generated surrogates;

Physiological wellness indication: a non-diagnostic indication generated from fused physiological, facial-appearance, and contextual features, such as a stress/anxiety indication, fatigue indication, nervous-behavior indication, activity-state indication, recovery-state indication, hydration-support indication, or other wellness-oriented interpretation;

### B. System Overview

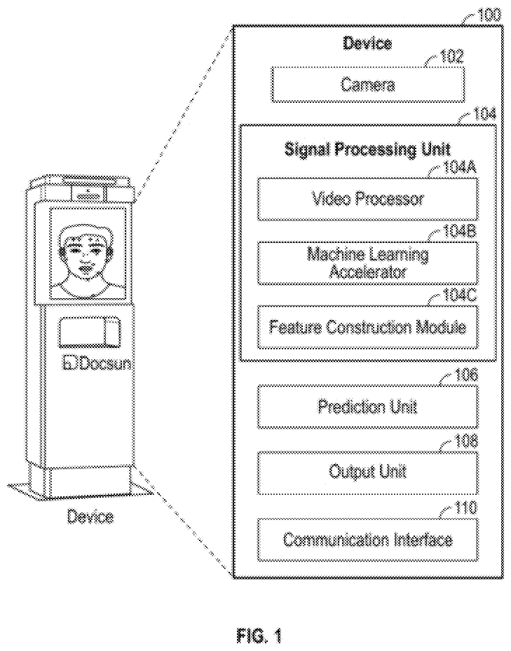

FIG. 1 illustrates an overall architecture of a non-contact physiological monitoring system including a monocular RGB video camera, an acquisition and quality-control pipeline, a feature fusion subsystem, a physiological prediction subsystem and a wellness retrieval subsystem-the prediction unit 106, and an output subsystem, in accordance with one embodiment of the present disclosure.

Referring generally to FIG. 1, one embodiment of the disclosed non-contact physiological monitoring device 100 includes a camera 102, a signal processing unit 104, a video processor 104A, a machine learning accelerator 104B, a feature construction module 104C, a prediction unit 106, an output unit 108, and a communication interface 110. In certain embodiments, the signal processing unit 104 is configured to perform one or more of acquisition, preprocessing, face detection, landmark detection, ROI selection, iPPG extraction, iBCG extraction, OCT-variation feature-map generation, and fused feature construction. In certain embodiments, the prediction unit 106 is configured to estimate pulse rate and one or more additional physiological parameters, and the output unit 108 is configured to present the estimated physiological parameters, uncertainty metrics, interpretability cues, and wellness indications locally or via the communication interface 110. In one embodiment, the camera captures facial video in a range of about 30 to 60 frames per second, although higher frame rates may also be used. Operation under ambient lighting is preferred because it permits opportunistic use on ordinary devices such as smartphones, tablets, laptops, in-vehicle cameras, kiosks, smart mirrors, or other consumer or professional equipment. The disclosed system does not require a physical OCT sensor, an interferometric OCT instrument, or direct-contact physiological sensors during inference, although such sensors may optionally be used during training, validation, calibration, or labeling.

The acquisition subsystem may perform exposure normalization, white-balance normalization, temporal synchronization, face detection, landmark detection, pose estimation, and skin masking. Candidate skin ROIs are proposed based on facial topology, vascular richness, pixel stability, pose visibility, and occlusion state. Rather than assuming that every facial area is equally useful, the system scores candidate ROIs using a temporal iPPG quality metric and retains the most stable pulsatile ROIs for subsequent analysis.

The physiological prediction subsystem may output pulse rate as a primary output and may optionally output heart-rate variability, respiration rate, blood oxygen saturation, blood-pressure values or trends, signal-quality estimates, or model uncertainty. In certain embodiments, the system further transforms those physiological outputs, together with appearance-derived facial features and user context, into textual contexts suitable for retrieval from a graph-augmented knowledge base and generation of evidence-grounded wellness indications.

### C. Facial Landmarking and Selection of Stable Pulsatile ROIs

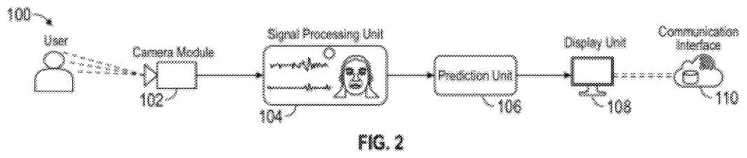

FIG. 2 illustrates an exemplary data-flow diagram in which facial video frames are converted into selected stable pulsatile ROIs, iPPG features, iBCG and facial micro-motion features, OCT-variation feature maps, fused feature vectors, predicted physiological parameters, and wellness indications, in accordance with one embodiment of the present disclosure.

Referring generally to FIG. 2, a subject or user may be positioned before a camera module 102. The camera module 102 may acquire facial video and provide image data to a signal processing unit 104. The signal processing unit 104 may identify facial landmarks, define candidate regions of interest, select stable pulsatile ROIs, and provide processed ROI data to a prediction unit 106. The prediction unit 106 may generate one or more physiological outputs, and the outputs may be provided to a display unit 108 or transmitted through a communication interface 110. In certain embodiments, the processing flow shown in FIG. 2 includes the extraction of iPPG signals, optional iBCG or facial micro-motion features, OCT-variation feature maps, fused feature vectors, predicted physiological parameters, and wellness indications.

In one embodiment, facial landmarks and facial regions are segmented using one or more geometric or machine-learning techniques. Suitable techniques include geometric triangulation over detected landmark points, dense face meshes, semantic face parsing, convolutional landmark detectors, transformer-based landmark detectors, and hybrid approaches that combine geometric constraints with learned segmentation. Candidate ROIs may include the forehead, central forehead, left cheek, right cheek, nasal bridge, malar regions, periorbital-adjacent skin, chin, jawline, or subdivided facial patches.

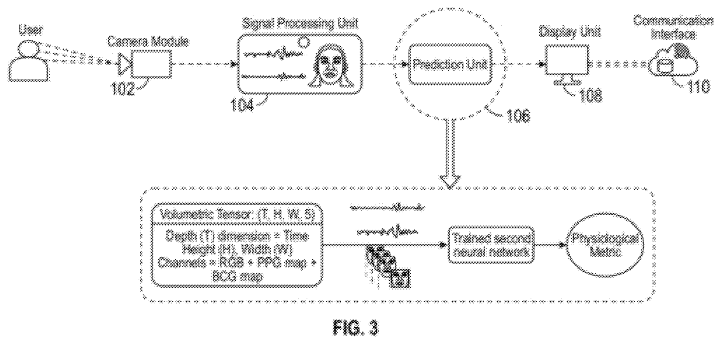

FIG. 3 illustrates an exemplary mathematical and algorithmic framework for temporal iPPG quality scoring, feature fusion, uncertainty estimation, and prediction of pulse rate and additional physiological parameters, in accordance with one embodiment of the present disclosure.

Referring generally to FIG. 3, image data acquired by the camera module 102 may be processed by the signal processing unit 104 and prediction unit 106 to generate one or more health-related outputs. In certain embodiments, the machine learning accelerator 104B may execute a trained image-recognition or physiological prediction model configured to process ROI data, iPPG-related data, BCG or iBCG-related data, and derived image features. The model may identify physiological signal patterns, determine health anomalies or wellness indications, and provide outputs through the display unit 108 or communication interface 110. The framework of FIG. 3 may include temporal iPPG quality scoring, ROI selection, feature fusion, uncertainty estimation, and prediction of pulse rate or additional physiological parameters.

The temporal iPPG signal quality metric may be computed for each candidate ROI over a defined temporal window. In one exemplary implementation, a raw candidate ROI trace is generated by aggregating pixel values from one or more color channels after motion compensation and skin masking. The trace is then band-limited or otherwise processed to identify a cardiac band, and one or more of the following components are computed: temporal signal-to-noise ratio; harmonic-to-noise ratio; spectral concentration within an expected pulse band; periodicity score; inter-window consistency; peak morphology score; motion-penalty score; illumination-stability score; and cross-ROI coherence score.

A combined quality score may then be generated, for example as Q(r)=w1*SNR(r)+w2*H(r)+w3*P(r)+w4*C(r)−w5*M(r)−w6*L(r), where SNR(r) denotes temporal signal-to-noise ratio for ROI r, H(r) denotes a harmonic stability metric, P(r) denotes periodicity, C(r) denotes coherence with one or more neighboring or reference ROIs, M(r) denotes motion contamination, L(r) denotes illumination instability, and w1 through w6 are learned or predetermined weights. The foregoing expression is exemplary and non-limiting; any rule-based, probabilistic, or learned quality metric that selects stable pulsatile ROIs may be used.

In preferred embodiments, ROI selection is adaptive over time. If a previously selected ROI degrades due to head rotation, transient shadow, hand occlusion, facial expression, eyewear reflection, or other disturbance, the system may down-weight that ROI and select one or more alternate ROIs from the candidate pool. In one embodiment, multiple ROIs are retained and ranked, and downstream inference uses either the top-ranked ROI, an attention-weighted combination of ROIs, or a confidence-weighted ensemble across ROIs.

### D. Extraction of iPPG Signals and Temporal Features

Once stable pulsatile ROIs have been selected, the system extracts imaging photoplethysmography signals and temporal features from the retained ROIs. In one embodiment, ROI pixel values are normalized for global intensity drift, local pose change, and non-skin contamination. Color-space transforms, adaptive detrending, temporal filtering, chrominance projection, independent component analysis, learned temporal encoders, or combinations thereof may be used to derive candidate iPPG waveforms.

The extracted temporal features may include pulse waveform amplitude; beat-to-beat amplitude variability; fundamental frequency; harmonics; harmonic ratios; temporal signal-to-noise ratio; peak sharpness; rise time; decay time; systolic-diastolic asymmetry; pulse regularity; inter-beat interval sequences; low-frequency and high-frequency components; and any learned latent representation generated from the ROI time series. In some embodiments, features are computed per ROI and then fused; in other embodiments, ROI sequences are provided directly to one or more neural encoders that learn the temporal features implicitly.

The extracted iPPG signals are not directly used for pulse-rate estimation, respiration-rate estimation, oxygen-saturation inference, heart-rate variability estimation, pulse morphology analysis, or quality gating. In certain embodiments, the temporal feature extraction stage also generates signal-quality flags or window-level confidence scores that are carried forward to later processing stages.

### E. Inference of OCT-Variation Feature Maps

A central aspect of the disclosed system is the inference of an OCT-variation feature map for each selected ROI by executing a trained convolutional neural network on the facial video frames. The term “OCT-variation feature map” does not require that the output be an actual OCT scan or a one-to-one reconstruction of interferometric data. Rather, in preferred embodiments it is a learned representation that emulates structural or coherence-depth patterns characteristic of OCT imaging, such as subsurface stratification, localized coherence variation, vessel-depth surrogates, tissue-thickness surrogates, perfusion-layer organization, or other depth-sensitive or structure-sensitive latent patterns.

In one training approach, an RGB-video encoder is trained using paired or semi-paired development data that includes RGB facial video and a target representation derived from OCT, OCT-like surrogates, perfusion maps, structured-light data, ultrasound-derived proxies, or subdermal proxy feature signals. The target representation may be a full image, a reduced feature map, a vessel-confidence map, a layer-transition map, a depth-coded latent tensor, or any transformed representation that preserves information useful for physiological prediction. During inference, the trained encoder receives only RGB video and outputs the OCT-variation feature map without use of any physical OCT hardware.

In another training approach, explicit OCT training targets are not required for every example. Instead, the network may be weakly supervised or self-supervised using cross-modal consistency losses, temporal-consistency losses, contrastive learning, masked-frame reconstruction, teacher-student distillation, surrogate structural losses, or physiological-consistency constraints. For example, the network may be trained to generate latent maps whose inter-region relationships and temporal stability improve downstream prediction of pulse rate, blood-pressure-related features, or heart-rate variability, when combined with extracted physiological signals, while maintaining consistency under appearance-preserving augmentations.

The OCT-variation feature map may be generated per frame, per temporal window, or per ROI sequence. In some embodiments, a stack of OCT-variation feature maps over time is created and aligned with the extracted iPPG and motion-derived features. In other embodiments, a temporal aggregator compresses the sequence into a lower-dimensional latent map before fusion.

### F. Motion-Derived Features, iBCG, and Facial Micro-Motion

In preferred embodiments, the selected ROIs are further processed to extract motion-derived features including imaging ballistocardiography signals and facial micro-motion features. One suitable implementation tracks sub-pixel motion of facial feature points, skin texture patches, or dense optical-flow vectors over time. Displacement sequences may be differentiated to obtain velocity and acceleration signals, and one or more periodic components synchronized to cardiac ejection may be isolated.

Motion-derived features may include raw or filtered displacement trajectories; velocity and acceleration waveforms; periodic motion energy; phase-coherent motion maps; J-wave, H-wave, or I-wave candidate landmarks; ejection-related onset markers; cross-ROI motion lag; motion-spectral signatures; and learned latent representations derived from the motion fields. In one implementation, iBCG signals are estimated from displacement traces anchored to stable facial structures and corrected for rigid head motion using a global head-pose model or background-stabilization transform.

The iBCG and iPPG modalities are complementary. The iPPG features primarily encode volumetric hemodynamics, blood-volume pulsation, and pulse morphology, whereas the iBCG or other micro-motion features encode mechanical recoil, tissue displacement, and pulse-propagation timing. Fusing these modalities can improve robustness when either the color signal or the motion signal is degraded, and can improve estimation of cardiovascular parameters such as pulse rate, beat-to-beat variability, respiratory coupling, and blood-pressure-related indices.

In some embodiments, the system computes cross-modal features between iPPG and iBCG, such as phase delay, pulse-transit surrogates, time-to-peak differences, coherence scores, wavelet coupling, or learned cross-attention weights. These cross-modal features may be used directly in blood-pressure estimation, cardiovascular-state estimation, or uncertainty estimation.

### G. Fused Feature Vector and Volumetric Tensor Construction

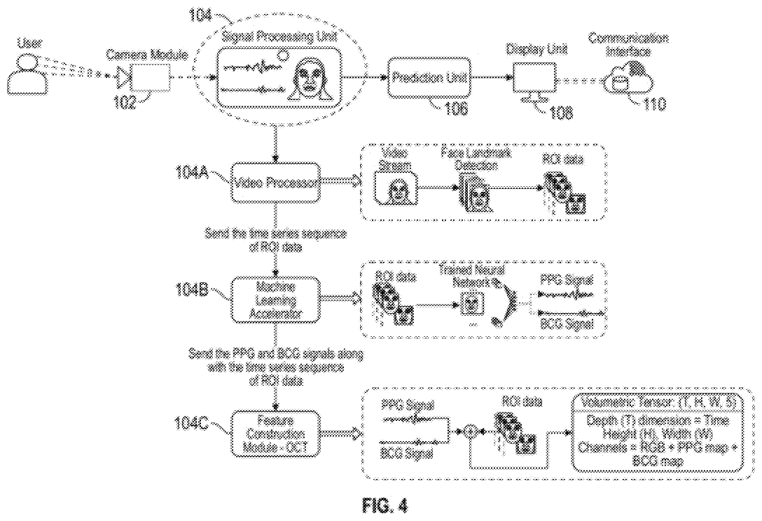

FIG. 4 illustrates an exemplary feature-construction module in which optical, mechanical, and contextual facial features are assembled into a fused vector and/or volumetric tensor for downstream inference, in accordance with one embodiment of the present disclosure.

Referring generally to FIG. 4, video or still-image data captured by the camera module 102 may be provided to the signal processing unit 104. The video processor 104A may process a still shot or video sequence of ROI images, and the machine learning accelerator 104B may process RGB image data, OCT-variation data or OCT-derived training targets, iPPG data, and BCG or iBCG data. The feature construction module 104C may combine PPG or iPPG signals, BCG or iBCG signals, facial image features, OCT-variation feature maps, and other ROI-derived features to generate a fused feature representation. In certain embodiments, the feature construction module 104C constructs a volumetric tensor having a temporal dimension corresponding to progression across video frames and feature channels corresponding to optical, mechanical, OCT-variation, and contextual features. The prediction unit 106 may process the fused representation to generate physiological predictions, which may be displayed through the display unit 108 or transmitted through the communication interface 110.

The optical features, temporal features, OCT-variation feature maps, and motion-derived features are then combined to generate a fused feature vector. In one embodiment, features from each retained ROI are aligned in time and concatenated. In another embodiment, an attention mechanism learns per-feature and per-ROI weights before fusion. In still another embodiment, the fused representation is organized as a volumetric tensor.

A non-limiting volumetric tensor may include: a temporal dimension corresponding to progression across video frames or temporal windows; two spatial dimensions corresponding to the coordinates of the selected ROI or a canonical face-aligned patch; and one or more feature channels corresponding to RGB-derived signals, skin masks, temporal iPPG features, harmonics, temporal signal-to-noise ratio, motion-derived features, iBCG features, OCT-variation feature maps, gradient maps, quality metrics, or contextual facial features.

Treating time as a depth-like dimension permits the system to analyze spatiotemporal relationships in a manner analogous to volume processing, while still operating on ordinary RGB video. The volumetric tensor may be populated on a sliding window basis, for example over windows of about 0.5 seconds to about 20 seconds, and may be updated continuously during runtime. The disclosed representation is especially useful for systems configured to employ convolutional layers followed by transformer layers with self-attention and positional encoding.

### H. Physiological Prediction Model and Uncertainty Estimation

The fused feature vector or volumetric tensor is provided to a trained model that predicts pulse rate and, optionally, one or more additional physiological parameters. In one embodiment, the trained model comprises one or more convolutional layers configured to extract local spatial and spatiotemporal features from the volumetric tensor, followed by one or more transformer layers with self-attention and positional encoding configured to capture longer-range dependencies across time, across ROIs, or across modalities.

The trained model may include a shared trunk and one or more prediction heads. A first prediction head may output pulse rate. Additional heads may output heart-rate variability metrics, respiration rate, blood oxygen saturation, blood-pressure values, and uncertainty. In one embodiment, uncertainty is estimated using a probabilistic inference mechanism including an ensemble of models, Monte Carlo dropout, Bayesian last-layer inference, heteroscedastic variance heads, quantile regression, or any combination thereof.

The system may output an uncertainty metric together with the physiological parameter value, such as a confidence interval, variance, percentile band, credibility score, quality flag, or pass/fail threshold. Windows or ROIs associated with poor quality may be rejected, down-weighted, or reported as low-confidence. This permits safer operation in real-world settings where lighting, motion, pose, and skin visibility vary over time.

In one embodiment, the volumetric tensor is processed by paired transformer encoders or by parallel transformer branches that generate latent variable outputs for different modalities, ROIs, or time scales. Those latent variable outputs may be concatenated, compared, or cross-attended and then fed to a downstream machine-learning model, which may itself include convolutional layers and transformer layers with self-attention and positional encoding, to correlate the latent variable outputs to physiological wellness indications selected from stress/anxiety, fatigue, nervous behavior, and activity monitoring. In preferred embodiments, the downstream wellness model operates on latent variables together with retrieved graph evidence or textual contexts so that the wellness indication remains grounded in measured physiological and appearance evidence.

In one embodiment, the output subsystem presents the predicted pulse rate together with an interpretability cue identifying at least one ROI used for inference. The interpretability cue may be an outlined ROI, a ranked list of contributing ROIs, a heat map overlay, or an attention map superimposed on the face image. In another embodiment, a remote system receives both the prediction and the interpretability cue for review or audit.

### Model Training, Reference Signals, and Data Preparation

To support the claimed architecture, the disclosed models may be trained using facial video sequences correlated with ground-truth physiological signals selected from electrocardiogram data, photoplethysmography data, pulse oximetry data, respiratory references, blood-pressure references, and subdermal proxy feature signals. Reference signals may be obtained from contact or near-contact instruments during development or validation even though such instruments are not required during inference.

In one exemplary development workflow, synchronized development data are collected from a diverse population over different skin tones, age groups, poses, ambient-light conditions, activity states, and device types. Each training record may include facial video, camera metadata, optional environmental metadata, ECG or reference pulse signals, pulse oximeter waveforms or SpO2 readings, respiratory reference signals, cuff-based blood pressure readings or arterial waveform data, and optional subdermal proxy targets. The system may also use weakly labeled or unlabeled facial video for pretraining or domain adaptation.

Training data may be prepared by face detection, face alignment, skin masking, temporal synchronization, ROI extraction, signal-quality labeling, and data augmentation. Non-limiting augmentations include brightness shifts, color jitter, compression artifacts, motion blur, simulated pose changes, partial occlusion, frame dropping, temporal warping, and domain randomization across camera characteristics. In preferred embodiments, augmentations preserve physiologically relevant temporal structure while increasing robustness to nuisance factors.

Different loss functions may be combined. A pulse-rate loss may use absolute error or frequency-domain error; a waveform-consistency loss may align predicted latent cardiac cycles with reference cycles; a blood-pressure loss may use regression; a cross-modal coherence loss may encourage consistent relationships between iPPG and iBCG; an uncertainty-calibration loss may align predicted confidence with observed error; and an OCT-variation consistency loss may preserve structural coherence across adjacent frames or augmentations.

In some embodiments, subject-normalization or session-normalization is employed. For example, amplitude features may be normalized to a personal baseline within a session, or demographic and environmental confounders may be reduced through domain-adversarial learning, stratified training, or metadata-conditioned normalization layers.

### Blood-Pressure and Cardiovascular Parameter Estimation

In one preferred embodiment, blood pressure is estimated from a combination of optical and mechanical information extracted from the facial video. The disclosed system does not require that blood pressure be estimated from a single color channel alone. Rather, the system may use: iPPG pulse morphology; inter-ROI phase difference; pulse-transit surrogates; pulse-wave velocity surrogates; iBCG onset timing; ejection-related motion signatures; waveform harmonic ratios; respiratory modulation; and learned latent variables from the fused spatiotemporal model.

In one non-limiting example, the system identifies a mechanical onset marker from iBCG-like motion features and an optical upstroke or peak from iPPG features, and the time relationship between those signals contributes to a pulse-transit-related feature. In another example, the system uses phase differences between two or more facial ROIs to estimate propagation-related information. In further embodiments, the model learns blood-pressure-sensitive latent variables directly from the volumetric tensor without requiring explicit hand-engineered transit times as intermediate outputs.

Heart-rate variability may be derived from beat-to-beat interval timing estimated from one or more optical or fused waveforms. Respiration rate may be inferred from slower modulation of pulse amplitude, subtle motion variation, or learned respiratory latent variables. Blood oxygen saturation may be inferred from color-dependent pulse features, waveform shape, or learned channel interactions. Because all of these outputs share common video evidence, the system may train them in a multi-task regime that improves generalization and cross-parameter consistency.

In some embodiments, the system outputs numeric estimates. In other embodiments, the system outputs trend classes, confidence bands, healthy/caution/abnormal ranges, or change-over-baseline indicators. The output form may depend on the quality of the captured signal, the intended use setting, the available training labels, or regulatory design choices.

### Facial Appearance Features for Wellness Indications

To support wellness-oriented inferences, the disclosed system may extract a second class of facial features that are not limited to periodic pulse signals. These may be referred to as facial appearance features, wellness support features, or contextual facial features. In one embodiment, such features are derived from one or more face-aligned images, ROI-specific image patches, temporal averages, temporal changes, or learned embeddings produced by the same or a separate vision encoder.

Non-limiting facial appearance features include erythema or localized redness; pallor or relative loss of visible perfusion; oiliness or sebum-related surface gloss; dryness or low-hydration appearance; uneven tone; dullness; pore prominence; roughness; flaking; periorbital darkness; periorbital puffiness or edema-like fullness; asymmetry of color or texture across left and right facial regions; facial thermal or pseudo-thermal surrogates if derived from RGB video or optional multimodal sources; blink frequency; eyelid opening stability; and expression-related tension features.

These features may be measured as absolute values, percentile values, relative values, ratios between facial subregions, subject-specific baseline deviations, session-normalized z-scores, or ordinal categories such as low, moderate, or elevated. For example, erythema may be represented by a malar redness index, oiliness by a T-zone gloss index, dryness by a texture-and-low-specularity hydration appearance score, and periorbital change by darkness and puffiness indices relative to the subject's own cheek baseline.

The facial appearance features are preferably not treated as standalone diagnoses. Instead, they are used as evidence features that interact with physiological outputs and contextual inputs to support higher-level wellness indications. For example, elevated facial redness together with elevated pulse rate and reduced heart-rate variability may support a stress-related or exertion-related wellness indication, whereas periorbital puffiness, low activity state, and altered respiratory pattern may support a fatigue or recovery-related wellness indication. Similarly, high sebum gloss, enlarged pore prominence, and increased redness may be routed to a skin barrier, inflammation, or routine-selection branch of the wellness reasoning module.

### Conversion of Measured Features into Textual Contexts

In one embodiment, measured physiological parameters and facial appearance features are transformed into normalized textual contexts before retrieval. This stage converts continuous and categorical outputs into a compact, machine-readable textual representation that can be consumed by a retrieval template, a graph traversal engine, and/or a domain-adapted language model.

A non-limiting physiological textual context may include strings such as: “pulse_rate=elevated”, “pulse_rate_trend=upward”, “hrv=suppressed”, “respiration_rate=mildly_elevated”, “spo2=within_expected_range”, “blood_pressure=upward_trend”, or “confidence=moderate”. A non-limiting facial textual context may include strings such as: “malar_erythema=moderate”, “t_zone_oiliness=elevated”, “periorbital_darkness=increased”, “periorbital_puffiness=present”, “hydration_appearance=reduced”, “texture_roughness=high”, “pore_prominence=elevated”, or “facial_pallor=mild”.

A non-limiting user context may include device type, age band, sex, known skin type, self-reported sleep quality, recent activity, time of day, ambient temperature, ambient humidity, caffeine intake, medication status, menstrual-cycle phase, or other metadata voluntarily provided by the user or inferred from the session. In preferred embodiments, user context is optional and is separated from direct physiological evidence so that the reasoning engine can distinguish measured evidence from background context.

Thresholding may be global, demographic-specific, skin-tone-aware, or personalized. In one embodiment, textual contexts are generated by comparing each feature against a subject baseline established during a low-motion, reference-quality window. In another embodiment, the thresholds are population-based and stratified by demographic and environmental factors. In yet another embodiment, a learned discretizer or classifier generates the textual contexts directly from the latent feature space.

### Curated Knowledge Base and GraphRAG Wellness Retrieval

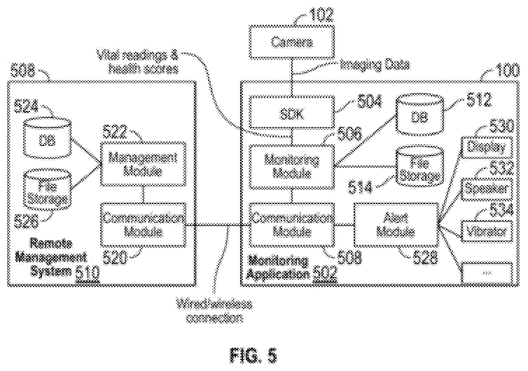

FIG. 5 illustrates an exemplary monitoring and knowledge-service configuration in which predicted physiological parameters and textual contexts are transmitted to a knowledge retrieval service configured to query a curated graph-based and vector-based knowledge base, in accordance with one embodiment of the present disclosure.

Referring generally to FIG. 5, a monitoring application 502 may be implemented on or in communication with a device 100 having a camera 102. The monitoring application 502 may include an SDK 504, a monitoring module 506, and a communication module 508. The monitoring application 502 may store or retrieve information from a database 512 and file storage 514, and may provide outputs through a display 530, speaker 532, vibrator 534, or alert module 528. In certain embodiments, the monitoring application 502 communicates through a wired or wireless connection with a remote management system 510 having a communication module 520, a management module 522, a database 524, and file storage 526. Predicted physiological parameters, vital readings, health scores, uncertainty values, interpretability cues, and textual contexts may be transmitted to the remote management system 510 or another knowledge service for graph-based retrieval, vector-based retrieval, evidence ranking, wellness indication generation, or remote monitoring.

In preferred embodiments, physiological wellness indications are not produced by an ungrounded language model alone. Instead, the system uses a curated, provenance-aware knowledge base configured for graph-based retrieval, vector-based retrieval, or a hybrid GraphRAG framework. The knowledge base may be implemented locally on device, in a secure cloud environment, or in a hybrid deployment.

In one implementation, the knowledge base has at least three cooperating layers. A first layer is a curated domain graph that stores manually reviewed, finite, relatively stable knowledge. A second layer is an evidence graph constructed from text corpora, for example by extracting entities, relations, and provenance from selected documents. A third layer is a vector index or semantic retrieval layer over chunked evidence passages. The curated graph acts as a precision anchor, the evidence graph acts as a multi-hop evidence layer, and the vector index acts as a semantic fallback when entity matching is incomplete.

The curated domain graph may include node types such as VitalSignal, FacialFeature, WellnessIndication, ActivityState, RecoveryState, Routine, Supplement, DietPattern, ContextFactor, Contraindication, SafetyRule, and EvidenceSource. Edge types may include MAY_INDICATE, ASSOCIATED_WITH, CORRELATES_WITH, MAY_BE_IMPROVED_BY, MAY_BE_EXACERBATED_BY, CONTRAINDICATED_FOR, MODULATED_BY, and SUPPORTED_BY. For example, an elevated_hrv_suppression node may connect to stress_related_state via MAY_INDICATE; periorbital_puffiness may connect to sleep_disruption or fluid_shift hypotheses via ASSOCIATED_WITH; and a breathing_routine node may connect to stress_related_state via MAY_BE_IMPROVED_BY.

The evidence graph may be created by chunking and processing selected textual sources and extracting research entities and typed relations. The extracted nodes may represent physiological variables, appearance features, biological processes, symptoms, environmental modifiers, interventions, nutrients, ingredients, activities, and contraindications. Each extracted edge may be linked back to one or more source chunks so that the system can provide evidence-backed generation.

The vector index may be built over the same evidence chunks and may support semantic retrieval when the textual contexts do not match graph entities exactly. In one embodiment, graph retrieval is attempted first, followed by vector fallback if the number, quality, or diversity of graph hits falls below a threshold.

### Exemplary Knowledge-Base Curation Workflow

The knowledge base may be curated from multiple source tiers. In one preferred workflow, Tier 1 sources include peer-reviewed papers, review articles, clinical guidelines, society statements, regulatory or standards documents, textbooks, and internally curated clinical or domain notes. Tier 2 sources may include validated care pathways, de-identified clinical notes or coaching notes that have been scrubbed of protected information and manually reviewed, structured educational content, and internal annotation guides. Tier 3 sources may include fitness blogs, wellness coach notes, athlete recovery notes, or lifestyle resources that are useful for low-risk recommendation phrasing but are marked with lower evidentiary weight and are not used alone to support high-risk medical warnings.

A source-ingestion workflow may include: source discovery; rights verification; metadata capture; conversion to plain text; chunking; deduplication; ontology mapping; named-entity extraction; relation extraction; provenance assignment; evidence scoring; human review; graph population; vector embedding; and regression tests using benchmark retrieval questions. In one embodiment, every chunk receives metadata such as source type, publication date, evidence tier, topic labels, contraindication labels, and one or more confidence or review fields.

Exemplary search queries used during source discovery may include topic-specific research queries such as: “remote photoplethysmography heart rate variability facial video”; “imaging ballistocardiography blood pressure micro-motion facial”; “facial erythema dehydration skin barrier”; “sebum stress cortisol skin”; “periorbital edema sleep deprivation”; “facial pallor perfusion fatigue”; “breathing exercise HRV stress recovery”; “nutrition hydration skin barrier recovery”; “blood pressure variability sodium sleep activity”; and “exercise recovery heart rate variability wellness”. Additional query families may target routine recommendations, supplements, diet patterns, contraindications, and contextual modifiers.

In one embodiment, the curation workflow intentionally separates evidence used for medical or physiological associations from evidence used for low-risk behavioral or wellness suggestions. For example, peer-reviewed literature and guidelines may support the association between certain measured patterns and stress, fatigue, or cardiovascular load, while lower-tier wellness sources may contribute optional phrasing or examples for hydration routines, breathing exercises, recovery routines, supplement timing, or diet suggestions. The lower-tier material is tagged as such and may be filtered, down-weighted, or excluded depending on the operating mode.

The curated knowledge base may be updated periodically. Before deployment of an update, benchmark prompts may be run to confirm that retrieval remains grounded, that contraindications are respected, and that the system does not produce recommendations that are unsupported by the retrieved evidence.

### Retrieval Template and Evidence-Grounded Language Model

In one embodiment, the normalized physiological textual context, facial textual context, and user context are inserted into a retrieval template. A non-limiting template may request retrieval of: candidate wellness indications, plausible contributing factors, lifestyle recommendations, diet suggestions, supplement suggestions, contraindications, evidence triplets, and explanatory source chunks. The retrieval system may prioritize sources that match both measured physiological state and observed facial appearance state.

A non-limiting template may include fields such as: physiological_context, facial_context, user_context, evidence_priority, contraindication_flags, and requested_output_mode. For example, a query object may include a physiological context indicating elevated pulse rate and reduced HRV; a facial context indicating moderate erythema and elevated T-zone oiliness; and a user context indicating poor sleep and recent exercise. The retrieval system may then gather evidence regarding exertion, heat load, sympathetic activation, skin-barrier stress, dehydration support, post-exercise recovery, and appropriate low-risk routines.

The retrieved graph facts and evidence chunks may then be passed to a domain-adapted language model. The language model may be a medical language model, a wellness-tuned language model, or another model configured for grounded summarization and reasoning. In preferred embodiments, generation is constrained such that the model is required to cite or internally rely upon retrieved evidence, follow system rules, respect contraindication nodes, and distinguish between measured outputs, inferred hypotheses, and low-confidence possibilities.

The resulting output may include one or more physiological wellness indications selected from stress/anxiety, fatigue, nervous behavior, and activity monitoring, as well as explanatory text and recommendations relating to routines, supplements, diet, hydration, rest, breathing exercises, activity adjustment, or follow-up monitoring. In one embodiment, the output is expressly non-diagnostic and framed as an evidence-grounded indication or recommendation. In another embodiment, if risk thresholds are exceeded, the system suppresses informal recommendations and instead prompts repeat capture, external measurement, or clinician review.

### Example Wellness Inference Scenarios

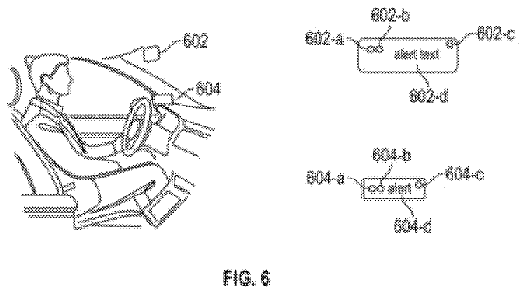

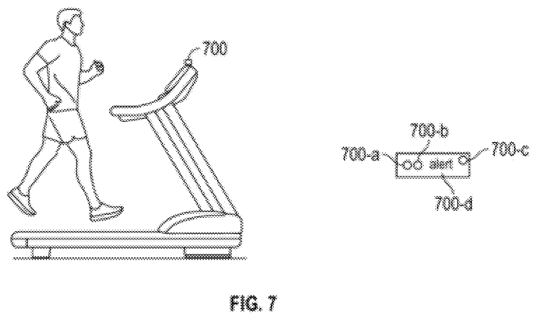

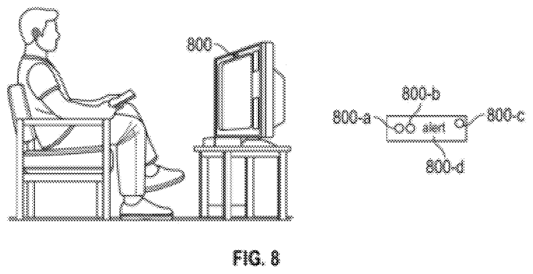

FIG. 6 illustrates an exemplary driver-monitoring embodiment in which a driver-facing monocular RGB camera operates as part of a vehicle or fleet-monitoring system.

Referring generally to FIG. 6, a driver-monitoring embodiment may include a vehicle-mounted camera or sensor 602 configured to acquire facial video from a driver. The system may estimate pulse rate, heart-rate variability, respiration-related features, facial micro-motion features, fatigue-related appearance features, stress-related indicators, or attention-related cues. Outputs may be provided through one or more alert interface components 602-a, 602-b, 602-c, and 602-d, or through a second alert interface 604 having alert interface components 604-a, 604-b, 604-c, and 604-d. In certain embodiments, the driver-monitoring implementation restricts outputs to safety-oriented alerts, capture-quality prompts, in-cabin notifications, or remote fleet-monitoring messages.

FIG. 7 illustrates an exemplary fitness or activity-monitoring embodiment in which the disclosed system operates in connection with exercise equipment, a smartphone, or a consumer wellness device.

Referring generally to FIG. 7, a fitness-monitoring embodiment 700 may acquire facial video from a subject during or after exercise, including through a camera associated with exercise equipment, a treadmill, a smartphone, or another consumer wellness device. The system may monitor pulse rate, respiratory state, recovery trend, facial erythema, perspiration-related surface sheen, heart-rate variability, blood-pressure-related trends, and activity-state indicators. Outputs may be provided through fitness alert interface components 700-a, 700-b, 700-c, and 700-d, and may include cool-down guidance, hydration support, activity pacing, or recovery-oriented wellness indications.

FIG. 8 illustrates an exemplary home or consumer-device embodiment in which the disclosed system is integrated into a display device, a smart mirror, or a personal-monitoring station.

Referring generally to FIG. 8, a home or consumer-device embodiment 800 may include a camera associated with a display, smart mirror, television, personal-monitoring station, mobile device, or other consumer device. The system may acquire facial video during ordinary user interaction and may output physiological estimates, uncertainty values, interpretability cues, and wellness indications through home-device alert interface components 800-a, 800-b, 800-c, and 800-d.

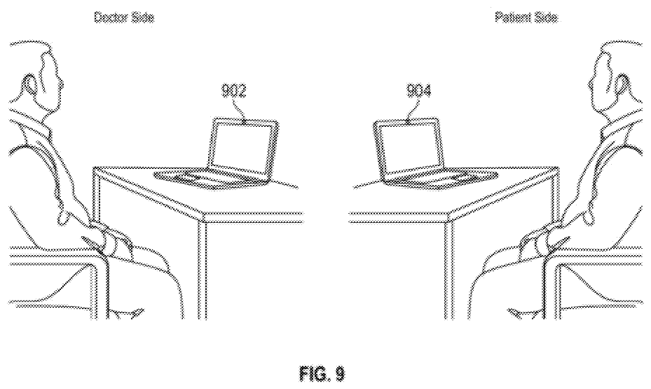

FIG. 9 illustrates an exemplary telehealth embodiment in which the disclosed system operates through a client device or care portal to provide real-time physiological and wellness indications during a remote encounter.

Referring generally to FIG. 9, a telehealth embodiment may include a doctor side 902 and a patient side 904. A patient-side device may capture facial video and generate physiological estimates, interpretability cues, uncertainty values, textual contexts, and wellness summaries. The doctor side 902 may receive one or more outputs for review during a remote encounter. In certain embodiments, the system provides an evidence-grounded summary while maintaining a distinction between measured physiological values, inferred wellness indications, and non-diagnostic recommendations.

In one example, a user performs a 15-second capture after a night of poor sleep. The system detects a stable pulse signal, reduced heart-rate variability, mildly elevated pulse rate, increased periorbital darkness, and visible periorbital puffiness. The wellness engine converts these into textual contexts and retrieves evidence relating to fatigue, sleep disruption, autonomic stress, hydration support, recovery routines, and activity pacing. The grounded language model then outputs a fatigue-related wellness indication together with low-risk recommendations such as hydration, a reduced-intensity activity recommendation, a breathing routine, and a suggestion to repeat the measurement after rest.

In another example, a subject completes exercise and initiates a measurement during cool-down. The system detects increased pulse rate, elevated respiratory rate, moderate facial erythema, and elevated sebum gloss or surface sheen. Based on user context indicating recent exercise, the retrieval engine prioritizes exertion, heat load, cardiovascular recovery, post-exercise hydration, and cool-down recommendations rather than unrelated inflammatory hypotheses. The system may therefore generate an activity-state indication and a recovery-oriented routine recommendation.

In another example, a subject in a driver-monitoring setting exhibits increasing pulse rate, reduced heart-rate variability, facial tension, and repeated shifts in attention or eyelid behavior over time. The system may classify an elevated stress or fatigue indication and route an alert to an in-cabin interface or remote monitoring service. In preferred embodiments, the operating mode may restrict outputs to safety-oriented messages rather than detailed lifestyle guidance.

In still another example, a telehealth platform uses the disclosed system to obtain an evidence-grounded wellness summary before or during a remote encounter. The summary may include measured pulse rate, an uncertainty score, a ranked list of contributing ROIs, a trend indicator, and one or more wellness indications, while also making the retrieved evidence available for audit.

### Apparatus Embodiments

In one apparatus embodiment corresponding to the disclosed method, a non-contact, non-invasive physiological monitoring apparatus comprises a monocular RGB video camera configured to capture a sequence of facial video frames of a subject, one or more processors operatively coupled to the camera, and a memory storing instructions that, when executed by the one or more processors, cause the one or more processors to perform operations including capturing facial video frames, selecting stable pulsatile ROIs using a temporal iPPG signal quality metric, extracting iPPG signals and temporal features, inferring OCT-variation feature maps, generating fused feature vectors, predicting pulse rate, and outputting the predicted pulse rate together with an interpretability cue.

In embodiments, the apparatus further extracts motion-derived features including iBCG and facial micro-motion features, constructs a volumetric tensor, applies a convolutional and transformer-based model with self-attention and positional encoding, estimates uncertainty, predicts additional physiological parameters including heart-rate variability, respiration rate, blood oxygen saturation, and blood pressure, and generates wellness indications using a retrieval-augmented reasoning module.

The apparatus may be implemented in whole or in part on a smartphone, tablet, laptop, desktop computer, embedded vehicle processor, edge appliance, kiosk, smart display, medical workstation, or hybrid client-cloud architecture. Some embodiments perform all primary inference on-device, while others offload selected retrieval or language-model functions to a remote service.

### Non-Transitory Computer-Readable Medium Embodiments

In another embodiment, a non-transitory computer-readable medium stores instructions that, when executed by one or more processors operatively coupled to a monocular RGB video camera, cause the one or more processors to perform operations comprising: capturing a sequence of facial video frames of a subject via the monocular RGB video camera operating at a frame rate in a range of about 30 frames per second to about 60 frames per second under ambient lighting; detecting candidate skin regions of interest (ROIs) by segmenting facial landmarks and regions using geometric heuristics or machine learning and by computing a temporal imaging photoplethysmography (iPPG) signal quality metric to select stable pulsatile ROIs; extracting iPPG signals and temporal features from the selected ROIs, the temporal features including pulse waveform amplitude, harmonics, and temporal signal-to-noise ratio; inferring an OCT-variation feature map for each selected ROI by executing a trained convolutional neural network on the facial video frames, wherein the trained convolutional neural network has been trained to map RGB-video time series to features that emulate structural or coherence-depth patterns characteristic of OCT imaging without using interferometric OCT hardware during inference; combining the iPPG signals, the temporal features, and the OCT-variation feature map to generate a fused feature vector; predicting, via a trained model, a pulse rate value from the fused feature vector; and outputting the pulse rate value together with an interpretability cue identifying at least one ROI used for inference. The computer-readable medium may be distributed across one or more memory devices and may be executed in part locally and in part remotely, provided that the operational result remains consistent with the disclosed embodiments.

### Detailed Exemplary Method

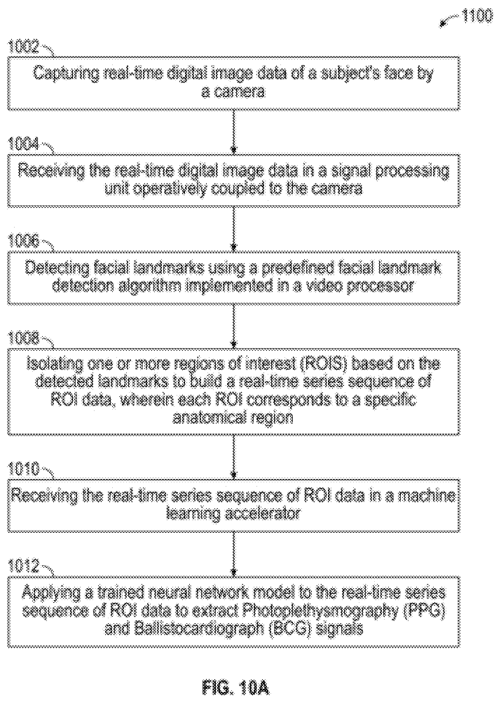

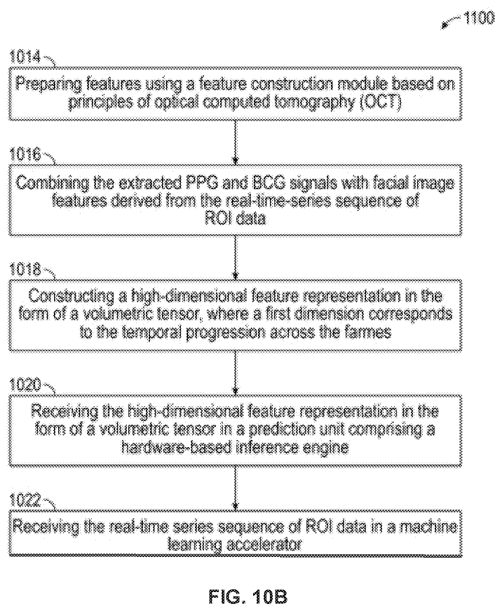

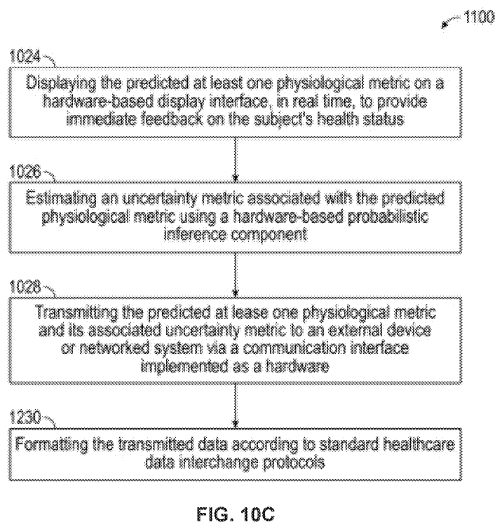

FIGS. 10A-10C illustrate an exemplary method for non-invasive physiological monitoring and wellness indication generation from facial video.

Referring generally to FIGS. 10A-10C, an exemplary method flow 1100 includes capturing real-time digital image data of a subject's face, receiving the image data in a signal processing unit operatively coupled to a camera, detecting facial landmarks, isolating one or more regions of interest, receiving ROI data in a machine learning accelerator, applying a trained neural-network model to the ROI data, preparing features based on OCT principles, combining iPPG signals and iBCG signals with facial image features, constructing a high-dimensional volumetric tensor, predicting at least one physiological metric, estimating uncertainty, transmitting the predicted metric to an external or networked system, and formatting transmitted data according to a healthcare data-exchange protocol.

At step 1002, the system captures, via a monocular RGB video camera operating in a range of about 30 to 60 frames per second under ambient lighting, a sequence of facial video frames of a subject.

At step 1004, the system detects a face, facial landmarks, and facial skin regions, and establishes candidate ROIs.

At step 1006, the system computes a temporal iPPG signal quality metric for each candidate ROI and selects one or more stable pulsatile ROIs.

At step 1008, the system extracts iPPG signals and temporal features from the selected ROIs, including pulse waveform amplitude, harmonics, and temporal signal-to-noise ratio.

At step 1010, the system executes a trained convolutional neural network on the facial video frames to infer an OCT-variation feature map for each selected ROI, without use of interferometric OCT hardware during inference.

At step 1012, the system extracts motion-derived features from the selected ROIs, including iBCG and facial micro-motion features.

At step 1014, the system combines the iPPG signals, temporal features, OCT-variation feature maps, and motion-derived iBCG features to generate a fused feature vector and, in some embodiments, a volumetric tensor.

At step 1016, the system predicts a pulse rate value and optionally one or more additional physiological parameters using a trained model comprising convolutional and transformer layers with self-attention and positional encoding.

At step 1018, the system estimates an uncertainty metric associated with one or more predicted physiological parameters.

At step 1020, the system outputs the pulse rate value together with an interpretability cue identifying at least one ROI used for inference and optionally outputs additional physiological parameters and uncertainty values.

At step 1022, the system optionally converts measured physiological parameters, facial appearance features, and user context into normalized textual contexts.

At step 1024, the system retrieves evidence from a curated graph-based and/or vector-based knowledge base using the textual contexts.

At step 1026, the system generates one or more physiological wellness indications and recommendations using a domain-adapted language model constrained by the retrieved evidence and safety rules.

At step 1028, the system transmits the predicted at least one physiological metric and its associated uncertainty metric to an external or networked system via a communication interface.

At step 1230, the system formats the transmitted data according to one or more standard healthcare data-exchange protocols.

### Additional Implementation Details

The disclosed embodiments may be implemented with different model sizes, latency budgets, and memory budgets. For example, a lightweight edge model may perform ROI selection, pulse-rate estimation, and uncertainty estimation on-device, while a larger remote model handles retrieval and wellness generation. Alternatively, all functions may execute on-device for privacy-sensitive deployments.

In some embodiments, the system includes a liveness or capture-authenticity module as part of acquisition quality control. Because authentic facial video generally exhibits physiologically plausible periodic optical and motion signatures, the system may reject frames or windows that appear replayed, synthetic, or non-physiological before computing wellness indications. Such quality control is optional and need not be claimed as a separate output to remain useful in the disclosed architecture.

The present disclosure contemplates numerous variations in network topology, feature ordering, and loss design. For example, the OCT-variation encoder may precede, follow, or operate in parallel with the iPPG encoder; the fusion block may use concatenation, gating, cross-attention, tensor products, low-rank fusion, or graph message passing; and the wellness retrieval engine may use deterministic rules, graph walks, hybrid graph-vector retrieval, or evidence reranking before language-model generation.

It should be understood that terms such as processor, engine, module, service, subsystem, or interface refer to physical computing structures configured to execute machine instructions, manipulate stored data, or control capture and inference operations. Unless explicitly stated otherwise, the operations described herein are performed by one or more processors executing instructions stored in non-transitory memory.

The foregoing description is intended to be illustrative and not limiting. Variations, combinations, substitutions, and refinements may be made without departing from the scope of the disclosure as defined by the appended claims.

### REFERENCE NUMERALS

100: device
102: camera
104: signal processing unit
104A: video processor
104B: machine learning accelerator
104C: feature construction module
106: prediction unit
108: output unit
110: communication interface
502: monitoring application
504: SDK
506: monitoring module
508: communication module
510: remote management system
512: database
514: file storage
520: communication module
522: management module
524: database
526: file storage
528: alert module
530: display
532: speaker
534: vibrator
602: driver-monitoring embodiment
602A-602D: first alert interface components
604: second alert embodiment
604A-604D: second alert interface components
700: fitness-monitoring embodiment
700A-700D: fitness alert interface components
800: home or consumer-device embodiment
800A-800D: home-device alert interface components
902: doctor side
904: patient side
1002-1028, 1230: method steps
1100: method flow

What i claim is:

1. A computer-implemented method for non-invasive estimation of a subject's pulse rate using a monocular RGB video camera, capturing, via the monocular RGB video camera operating in a range of about 30 to 60 frames per second under ambient lighting, a sequence of facial video frames of the subject; detecting candidate skin regions of interest (ROIs) by:
a. segmenting facial landmarks and regions using geometric heuristics or machine learning; and
b. computing a temporal imaging photoplethysmography (iPPG) signal quality metric to select stable pulsatile ROIs;
c. extracting iPPG signals and temporal features from the selected ROIs, the temporal features including pulse waveform amplitude, harmonics, and temporal signal-to-noise ratio (SNR);
d. inferring an OCT-variation feature map for each selected ROI by executing a trained convolutional neural network on the facial video frames, wherein the trained convolutional neural network has been trained to map RGB-video time series to features that emulate structural or coherence-depth patterns characteristic of OCT imaging, without using interferometric OCT hardware during inference;
e. combining the iPPG signals, the temporal features, and the OCT-variation feature map to generate a fused feature vector;
f. predicting, via a trained model, a pulse rate value from the fused feature vector; and
outputting, via a display or a remote system, the pulse rate value together with an interpretability cue identifying at least one ROI used for inference, wherein the method is performed by one or more processors executing instructions stored in non-transitory memory, and wherein no physical OCT sensor is used in any step of the method.

2. The method of claim 1, further comprising extracting, from the selected ROIs, motion-derived features comprising imaging ballistocardiography (iBCG) signals, and facial micro-motion features.

3. The method of claim 2, wherein generating the fused feature vector further comprises combining the motion-derived features with the iPPG signals, the temporal features, and the OCT-variation feature map.

4. The method of claim 3, wherein generating the fused feature vector comprises constructing a volumetric tensor in which one dimension corresponds to temporal progression across the facial video frames, two dimensions correspond to spatial coordinates of at least one selected ROI, and one or more feature channels correspond to the iPPG signals, the temporal features, the motion-derived features, and the OCT-variation feature map.

5. The method of claim 4, wherein the trained model comprises convolutional layers and transformer layers including self-attention and positional encoding configured to process the volumetric tensor to capture spatiotemporal dependencies across the facial video frames, and wherein the AI model is trained using facial video sequences correlated with ground-truth physiological signals selected from the group consisting of electrocardiogram data, photoplethysmography data, pulse oximetry data, and subdermal proxy feature signals.

6. The method of claim 5, wherein the latent variable outputs from the transformer encoder pairs is fed into the trained machine learning model comprising convolutional layers and transformer layers including self-attention and positional encoding to correlate the latent variable outputs to physiological wellness indications selected from the group consisting of but not limited to stress/anxiety, fatigue, nervous behavior, and activity monitoring.

7. The method of claim 1, further comprising estimating an uncertainty metric associated with the physiological parameter by applying probabilistic inference, an ensemble of models, and outputting the uncertainty metric together with the pulse rate value.

8. The method of claim 1, wherein the trained model is further configured to predict at least one additional physiological parameter selected from the group consisting of heart-rate variability, respiration rate, blood oxygen saturation, and blood pressure.

9. A non-contact, non-invasive physiological monitoring apparatus, comprising: a monocular RGB video camera configured to capture a sequence of facial video frames of a subject; one or more processors operatively coupled to the monocular RGB video camera; and a memory storing instructions that, when executed by the one or more processors, cause the one or more processors to: capture, via the monocular RGB video camera operating in a range of about 30 to 60 frames per second under ambient lighting, the sequence of facial video frames of the subject; detect candidate skin regions of interest (ROIs) by:
a. segmenting facial landmarks and regions using geometric heuristics or machine learning; and
b. computing a temporal imaging photoplethysmography (iPPG) signal quality metric to select stable pulsatile ROIs;
c. extract iPPG signals and temporal features from the selected ROIs, the temporal features including pulse waveform amplitude, harmonics, and temporal signal-to-noise ratio (SNR);
d. infer an OCT-variation feature map for each selected ROI by executing a trained convolutional neural network on the facial video frames, wherein the trained convolutional neural network has been trained to map RGB-video time series to features that emulate structural or coherence-depth patterns characteristic of OCT imaging, without using interferometric OCT hardware during inference;
e. combine the iPPG signals, the temporal features, and the OCT-variation feature map to generate a fused feature vector;
f. predict a pulse rate value from the fused feature vector;
g. output, via a display or a remote system, the pulse rate value together with an interpretability cue identifying at least one ROI used for inference, wherein no physical OCT sensor is used by the apparatus during inference.

10. The apparatus of claim 9, wherein the instructions, when executed by the one or more processors, further cause the one or more processors to extract, from the selected ROIs, motion-derived features comprising imaging ballistocardiography (iBCG) signals, and facial micro-motion features.

11. The apparatus of claim 10, wherein the instructions, when executed by the one or more processors, further cause the one or more processors to combine the motion-derived features with the iPPG signals, the temporal features, and the OCT-variation feature map to generate the fused feature vector.

12. The apparatus of claim 11, wherein the instructions, when executed by the one or more processors, further cause the one or more processors to construct a volumetric tensor in which one dimension corresponds to temporal progression across the facial video frames, two dimensions correspond to spatial coordinates of at least one selected ROI, and one or more feature channels correspond to the iPPG signals, the temporal features, the motion-derived features, and the OCT-variation feature map.

13. The apparatus of claim 12, wherein the trained model comprises convolutional layers and transformer layers including self-attention and positional encoding configured to process the volumetric tensor to capture spatiotemporal dependencies across the facial video frames, and wherein the AI model is trained using facial video sequences correlated with ground-truth physiological signals selected from the group consisting of electrocardiogram data, photoplethysmography data, pulse oximetry data, and subdermal proxy feature signals.

14. The apparatus of claim 9, wherein the instructions, when executed by the one or more processors, further cause the one or more processors to estimate an uncertainty metric associated with the pulse rate value by applying probabilistic inference, an ensemble of models, and to output the uncertainty metric together with the physiological parameter value, wherein the trained model is further configured to predict at least one additional physiological parameter selected from the group consisting of heart-rate variability, respiration rate, blood oxygen saturation, and blood pressure.

15. A non-transitory computer-readable medium storing instructions that, when executed by one or more processors operatively coupled to a monocular RGB video camera, cause the one or more processors to perform operations comprising: capturing, via the monocular RGB video camera operating in a range of about 30 to 60 frames per second under ambient lighting, a sequence of facial video frames of a subject; detecting candidate skin regions of interest (ROIs) by:
a. segmenting facial landmarks and regions using geometric heuristics or machine learning; and
b. computing a temporal imaging photoplethysmography (iPPG) signal quality metric to select stable pulsatile ROIs;
c. extracting iPPG signals and temporal features from the selected ROIs, the temporal features including pulse waveform amplitude, harmonics, and temporal signal-to-noise ratio (SNR);
d. inferring an OCT-variation feature map for each selected ROI by executing a trained convolutional neural network on the facial video frames, wherein the trained convolutional neural network has been trained to map RGB-video time series to features that emulate structural or coherence-depth patterns characteristic of OCT imaging, without using interferometric OCT hardware during inference;
e. combining the iPPG signals, the temporal features, and the OCT-variation feature map to generate a fused feature vector;
f. predicting, via a trained model, a pulse rate value from the fused feature vector; and
outputting, via a display or a remote system, the pulse rate value together with an interpretability cue identifying at least one ROI used for inference, wherein no physical OCT sensor is used during performance of the operations.

16. The non-transitory computer-readable medium of claim 15, wherein the operations further comprise extracting, from the selected ROIs, motion-derived features comprising imaging ballistocardiography (iBCG) signals, and facial micro-motion features.

17. The non-transitory computer-readable medium of claim 16, wherein generating the fused feature vector further comprises combining the motion-derived features with the iPPG signals, the temporal features, and the OCT-variation feature map.

18. The non-transitory computer-readable medium of claim 17, wherein generating the fused feature vector comprises constructing a volumetric tensor in which one dimension corresponds to temporal progression across the facial video frames, two dimensions correspond to spatial coordinates of at least one selected ROI, and one or more feature channels correspond to the iPPG signals, the temporal features, the motion-derived features, and the OCT-variation feature map.

19. The non-transitory computer-readable medium of claim 18, wherein the trained model comprises convolutional layers and transformer layers including self-attention and positional encoding configured to process the volumetric tensor to capture spatiotemporal dependencies across the facial video frames, and wherein the AI model is trained using facial video sequences correlated with ground-truth physiological signals selected from the group consisting of electrocardiogram data, photoplethysmography data, pulse oximetry data, and subdermal proxy feature signals.

20. The non-transitory computer-readable medium of claim 15, wherein the operations further comprise estimating an uncertainty metric associated with the pulse rate value by applying probabilistic inference, an ensemble of models, and outputting the uncertainty metric together with the physiological parameter value, wherein the trained model is further configured to predict at least one additional physiological parameter selected from the group consisting of heart-rate variability, respiration rate, blood oxygen saturation, and blood pressure.
